(19) United States
(12) Patent Application Publication
(10) Pub. No.: US 2020/0015854 A1
(43) Pub. Date: Jan. 16, 2020

(54) INTRAVAGINAL DEVICES FOR ENHANCED NATURAL INSEMINATION AND ASSOCIATED SYSTEMS AND METHODS

(71) Applicant: Robert Alan Jacoby, Castaic, CA (US)

(72) Inventor: Robert Alan Jacoby, Castaic, CA (US)

(21) Appl. No.: 16/509,473

(22) Filed: Jul. 11, 2019

Related U.S. Application Data
(60) Provisional application No. 62/697,374, filed on Jul. 12, 2018.

(51) Int. Cl. A61B 17/425; A61F 6/08

(52) U.S. Cl. CPC A61B 17/425; A61F 6/08

(57) ABSTRACT

Intravaginal devices for presenting ejaculate produced during sexual intercourse to the cervix while inhibiting exposure to the intravaginal environment and associated methods are disclosed herein. An intravaginal device configured in accordance with embodiments of the present technology can include, for example, a tubular body having a first concave surface that defines a first depression at which to collect ejaculate produced during sexual intercourse, a second concave surface that defines a second depression at which to present the ejaculate to the external os, and a flow channel defined by an inner surface of the tubular body extending between the first and second depressions. Because ejaculate deposited into the first depression travels to the second depression through the flow channel, exposure to the inhospitable conditions of the intravaginal environment is limited. Moreover, by guiding the ejaculate through the external os, the intravaginal device may serve to increase intrauterine sperm count.

### TECHNICAL FIELD

The present technology related generally to devices for aiding fertility. In particular, several embodiments of the present technology are related to intravaginal devices for enhancing the likelihood of natural insemination and donor sperm insemination and associated systems and methods.

### BACKGROUND

According to the National Institutes of Health (NIH), fifteen percent of couples are unable to conceive after twelve months of consistent, unprotected sexual intercourse. There are many potential reasons for reduced fecundity. Approximately one-third of these cases involve male infertility and approximately one-third of these cases involve female infertility. The remaining balance involves both male and female infertility. Conventional therapies for fertility problems typically include medication or surgery. However, these therapies can pose significant health risks. For example, medications may have undesirable and/or unpredictable side effects, while surgeries may pose risk of complications due to their invasive nature.

Other therapies (e.g., assisted reproductive treatment) may also require a level of invasiveness and expense that puts these therapies out of reach of many couples searching for fertility assistance. Artificial insemination, for example, involves the deliberate introduction of spermatozoa into the uterus or the cervix for the purpose of achieving a pregnancy through in vivo fertilization by means other than sexual intercourse. Compared with natural insemination (i.e., insemination by sexual intercourse), artificial insemination is more expensive, invasive, and risky. Moreover, artificial insemination often requires professional medical assistance and multiple visits to a healthcare facility.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure. Furthermore, components can be shown as transparent in certain views for clarity of illustration only and not to indicate that the illustrated component is necessarily transparent. The headings provided herein are for convenience only.

FIG. 1 is a perspective view of an intravaginal device for enhanced natural insemination in accordance with embodiments of the present technology.

FIG. 2A is a perspective view of an intravaginal device for enhanced natural insemination in accordance with other embodiments of the present technology.

FIG. 2B is a side cross-sectional view of the intravaginal device of FIG. 2A.

FIG. 3 is a side cross-sectional view of an intravaginal device including a series of corrugations configured in accordance with embodiments of the present technology.

FIG. 4 is a side cross-sectional view of an intravaginal device including a valve configured in accordance with embodiments of the present technology.

FIG. 5 is a top view of a distal end portion of an intravaginal device configured in accordance with embodiments of the present technology.

FIG. 6 is a bottom view of a proximal end portion of an intravaginal device configured in accordance with embodiments of the present technology.

FIG. 7A is an isometric view of an intravaginal device in an expanded state configured in accordance with embodiments of the present technology.

FIG. 7B is an isometric view of the intravaginal device of FIG. 7A in a compressed state configured in accordance with embodiments of the present technology.

FIG. 8A is a side view of an intravaginal device for enhanced natural insemination in accordance with some embodiments of the present technology.

FIG. 8B is a bottom view of a proximal end portion of the intravaginal device of FIG. 8A in a collapsed state.

FIG. 8C is a bottom view of a proximal end portion of the intravaginal device of FIG. 8A in a natural state.

FIG. 9A is a side view of an intravaginal device for enhanced natural insemination in accordance with some embodiments of the present technology.

FIG. 9B is a side cross-sectional view of the intravaginal device of FIG. 9A.

FIG. 10 is an illustration of an intravaginal device deployed within the anatomy in accordance with embodiments of the present technology.

FIG. 11 is a series of side views illustrating a procedure for inserting an intravaginal device into the vaginal canal using an installation mechanism in accordance with embodiments of the present technology.

FIG. 12 is a block diagram illustrating a method for using an intravaginal device to enhance the likelihood of a pregnancy through natural insemination in accordance with embodiments of the present technology.

### DETAILED DESCRIPTION

The natural insemination process begins when spermatozoa, usually in the form of ejaculate, enters the vagina during sexual intercourse. The spermatozoa travel through the cervix and uterine cavity to the fallopian tubes, where they meet the ovum and fertilization takes place. According to recent studies, however, a vast majority of the spermatozoa never enters the uterus, and thus is not available for fertilization. This usually occurs due to a combination of factors. For example, the ejaculate may not be deposited close enough to the external orifice of the cervix (also referred to as the “external os”), and thus never passes through the external os, ejaculate may be deposited into the hostile environment of the fornix below, behind, or beneath the external os, ejaculate may exit through the vagina soon after ejaculation, and/or spermatozoa may die due to inhospitable conditions in the intravaginal environment.

Introduced here, therefore, are intravaginal devices designed to enhance the likelihood of natural insemination and associated systems and methods. Embodiments of the present technology are directed to systems, methods, and devices that address the unique challenges of enhancing the likelihood of a pregnancy through natural insemination in a safe, cost-effective manner. For example, some embodiments pertain to intravaginal devices configured to funnel ejaculate toward the external os, thereby bypassing the intravaginal environment. More specifically, an intravaginal device can comprise a tubular body that includes a first portion having a first concave surface that defines a first depression for receiving ejaculate during sexual intercourse, a second portion having a second concave surface that defines a second depression for presenting the ejaculate to the external os, and a flow channel defined by an inner surface of the tubular body extending between the first and second depressions. Upon deployment in the intravaginal environment, the second concave surface can be fit snugly around the opening of the cervix (also referred to as the “ectocervix” or “external os”). Ejaculate deposited into the first depression (e.g., during sexual intercourse) can travel to the second depression through the flow channel for presentation to the external os. Compared to conventional fertility therapies, embodiments of the present technology present ejaculate directly to the external os to inhibit loss of spermatozoa due to exposure to the inhospitable conditions in the intravaginal environment. In addition, intravaginal devices disclosed herein can easily force ejaculate through the external os when pressure is applied to the intravaginal device (e.g., by the penis during sexual intercourse), and/or ejaculate can be funneled in such a manner so as to inhibit incompetent deposition within the fornix. By guiding ejaculate directly into the cervix, the intravaginal devices may increase the intrauterine sperm count.

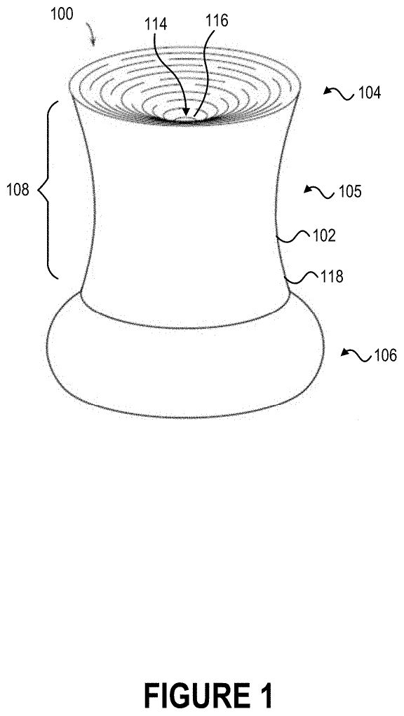

Specific details of several embodiments of the present technology are described herein with reference to FIGS. 1-12. Although many of the embodiments are described with respect to devices, systems, and methods for presenting ejaculate directly to the external os during sexual intercourse, other embodiments in addition to those described herein are within the scope of the present technology. For example, at least some embodiments of the present technology may be useful for delivering ejaculate to the cervix during assisted reproductive treatment procedures. It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

With regard to the terms “distal” and “proximal” within this description, unless otherwise specified, the terms can reference relative positions of portions of an intravaginal device and/or an associated delivery device with reference to a user. For example, in referring to an intravaginal device suitable to be introduced into the vagina, “distal” can refer to a first position close to where ejaculate is initially deposited during sexual intercourse (i.e., a position within the vagina and spaced apart from the external os), and “proximal” can refer to a second position close to where the ejaculate is presented to the external os.

### Selected Embodiments of Intravaginal Devices

FIG. 1 is a perspective view of an intravaginal cervical funnel (“IVCF”) or intravaginal device 100 designed to be inserted into the vagina prior to sexual intercourse in accordance with embodiments of the present technology. The intravaginal device 100 can serve as passive or semi-passive spermatozoa concentration mechanisms that increases the quantity of ejaculate that enters the cervix than would occur during unenhanced intercourse. By funneling or guiding ejaculate directly to the cervix (and thereby into the uterus), the intravaginal device 100 can significantly reduce the loss of spermatozoa and enhance the effectiveness of ejaculate deposited during sexual intercourse.

In the embodiment illustrated in FIG. 1, the intravaginal device 100 includes a tubular body 102 having a first or distal end portion 104, a medial portion 105, a second or proximal end portion 106, and a flow channel 114 defined by an inner surface 116 of the tubular body 102 and extending between the distal and proximal end portions 104 and 106. During sexual intercourse, ejaculate collects at the distal end portion 104, and then moves through the flow channel 114 where it is presented to the external os at the proximal end portion 106. As further described below, the tubular body 102 can be designed to fit snugly within the vaginal cavity during sexual intercourse. For example, the tubular body 102 may be sized larger than the vaginal canal such that an outer sidewall or surface 118 of the tubular body 102 contacts the walls of the vaginal canal when deployed. In such embodiments, the tubular body 102 can be anatomically designed to press against the walls of the vaginal canal near the cervix so that the outer surface 118 and the native tissue of the vaginal walls exert pressure against each other, thereby securing the intravaginal device 100 in place.

At least a portion of the tubular body 102 can be comprised of an elastomeric material, a sponge material, a foam material, combinations thereof, and/or other deformable materials. Because elastomeric materials, sponge materials, and foam materials are capable of deforming under pressure, tubular bodies comprised of such deformable materials can readily expand and compress along its lateral and longitudinal axis to accommodate anatomical structures of different sizes and shapes. In some embodiments, the entire tubular body 102 is comprised of a deformable foam material, while in other embodiments only a portion of the tubular body 102 is comprised of a deformable material and other portions of the tubular body 102 include rigid materials.

The tubular body 102 may be designed to include an elastic zone 108 (also referred to as a “crush zone”) that can be deformed under a force applied generally along a longitudinal axis of the intravaginal device 100. Such force may be applied, for example, by a penis on the distal end portion 104 during sexual intercourse. Upon application of certain pressures to the distal end portion 104, the elastic zone 108 can partially collapse or otherwise deform toward the proximal end portion 106 (e.g., to accommodate a force directed downward from the distal end portion 104 toward the proximal end portion 106).

As shown in FIG. 1, the outer surface 118 of the tubular body 102 may be non-linear in nature. For example, the tubular body 102 of FIG. 1 includes a stalk or body portion having a concave sidewall 118 that is arranged adjacent to an outwardly flared portion (e.g., the proximal end portion 106) designed to at least partially enclose or encircle the ectocervix or external os. The proximal end portion 106 can be hemispheric as illustrated in FIG. 1, may have a substantially straight outer surface, or may otherwise be flared outwardly from the tubular body 102 to encircle the external os. In other embodiments, the outer surface 118 of the tubular body 102 may be straight (e.g., a cylinder, rectangular prism, etc.), tapered, curved in a convex manner, asymmetrical, and/or have other suitable shapes for engaging the tissue walls of the vaginal cavity. In some embodiments, the proximal end portion 106 is a outwardly flared portion. In some embodiments, the outer surface 118 of the tubular body 102 is configured to be wetted with a lubricant for easier deployment.

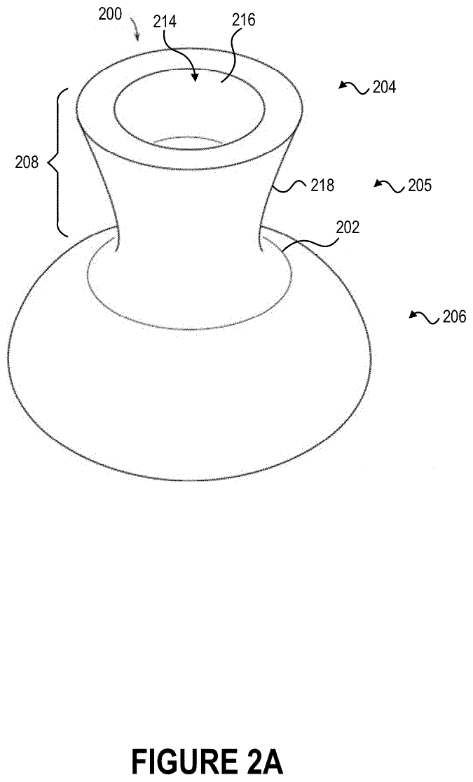

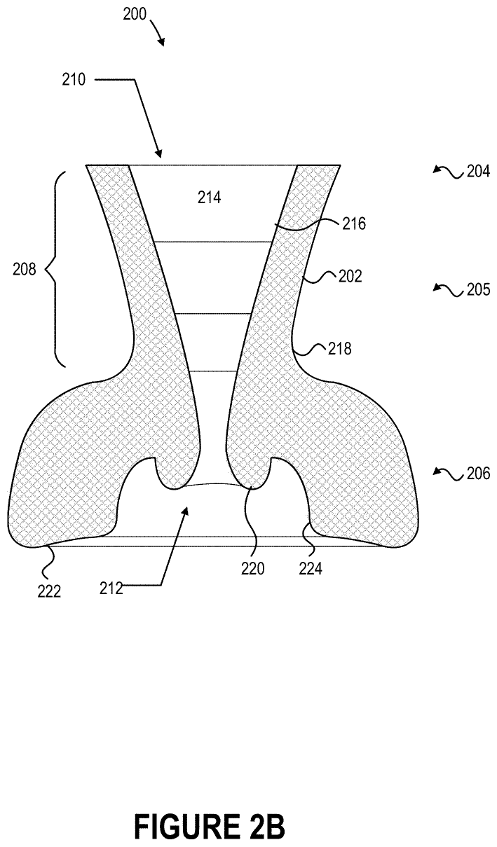

FIG. 2A is a perspective view of an intravaginal device 200 for enhanced insemination configured in accordance with embodiments of the present technology, and FIG. 2B is a side cross-sectional view of the intravaginal device 200 of FIG. 2A. The intravaginal device 200 includes several features at least generally similar to the intravaginal device 100 described with reference to FIG. 1 above. For example, the intravaginal device 200 includes a tubular body 202 having a distal end portion 204 configured to be positioned proximate to the vaginal opening, a proximal end portion 206 opposite the distal end portion 204 and configured to be positioned proximate to the cervix, a medial portion 205 spacing the distal and proximal portions 204 and 206 apart from each other, and a flow channel 214 defined by an inner surface 216 of the tubular body 202 and configured to transport ejaculate from the distal end portion 204 to the proximal end portion 206 where it can be presented to the external os of the cervix. The tubular body 202 can also include an elastic zone or region 208 that can at least partially collapse under pressure applied to the distal end portion 204 during intercourse. As shown in FIGS. 2A and 2B, rather than a concave sidewall, the tubular body 202 includes a stalk having a tapered sidewall 218 extending from the distal end portion 204 to the flared proximal end portion 206, which is sized and shaped to at least partially encircle the ectocervix or external os.

As shown in FIG. 2B, the intravaginal device 200 can further include a first opening 210 at the distal end portion 204 at which ejaculate can be naturally deposited during sexual intercourse or artificially deposited (e.g., via a syringe) and a second opening 212 at the proximal end portion 206 at which the ejaculate can be presented to the external os. When ejaculate is deposited at the first opening 210, it can travel to the second opening 212 through the flow channel 214. In various embodiments, the first opening 210 and/or the second opening 212 can be defined by or extend from depressions at the distal end portion 204 and/or the proximal end portion 206, respectively. In the embodiment illustrated in FIG. 2B, for example, the proximal end portion 206 includes a depression defined by an inner concave surface 224, and the second opening 212 is within or defined by the depression. In other embodiments, the depression may be defined by a surface that tapers from the primal-most terminus of the device 200 toward the channel 214 or a surface that has another suitable shape for collecting ejaculate and positioning it near the external os. The depression at the proximal end portion 206 may have a diameter of approximately 1.0-1.25 inches (25.4-31.75 mm). Often, the depression at the proximal end portion 206 is sized such that a gasket structure or an inner extension feature 220 located in the proximal end portion 206 can be secured around the ectocervix and the bulbous or thick sidewalls of the proximal end portion 206 are sized to at least partially fill the cervical fornix. Including one or more of these features inhibits exposure of ejaculate to the hostile intravaginal environment. As described in further detail below, similar to the proximally positioned depression, the distal end portion 204 of the tubular body 202 can include a depression defined by a concave or other inner surface of the device 200 and includes the first opening 210. The depression at the distal end portion 204 may have a diameter of approximately 1.0-1.25 inches (25.4-31.75 mm).

The flow channel 214 may have a diameter of approximately 0.125-0.250 inches (3.175-6.350 mm). In some embodiments, such as the embodiment shown in FIG. 2B, the flow channel 214 has a funnel design with a tapered sidewall 216 that narrows toward the proximal end portion 206. Thus, the flow channel 214 may decrease in width as proximity to the second opening 212 increases to guide the semen to the depression at the proximal end portion 206. For example, the flow channel 214 may have a diameter of approximately 1.0-1.25 inches (25.4-31.75 mm) at the first opening 210 and a diameter of approximately 0.124-0.250 inches (3.175-6.350 mm) at the second opening 212. In other embodiments, the cross-sectional dimension or width of the flow channel 214 remains constant along the length of the tubular body 202, decreases in width toward the proximal end portion 206, and/or has variations in width along its length.

In some embodiments, the tubular body 202 includes a gasket structure or an inner extension feature 220 at the proximal end portion 206 and defining the second opening 212. The extension feature 220 can be sized and shaped to fit over, around, or into the external os. When pressure is applied to the intravaginal device 200 at the distal end portion 204, the proximal end portion 206 may radially expand and the extension feature 220 may come within close proximity of the external os. For example, the edges of the extension feature 220 may extend around the cervical opening (external os) or into the endocervical canal via the external os while a proximal end surface of the tubular body 202 contacts the ectocervix and/or the vaginal wall around the ectocervix). Some embodiments of the intravaginal device 200 are designed such that the edges of the extension feature 220 are always in contact with the ectocervix.

The tubular body 202 may be sized to contact the walls of the vaginal canal when deployed. Thus, the proximal end potion 206 may have an outer maximum diameter of approximately 1.75-2.25 inches (44.45-57.15 mm). Such a design provides that, upon deployment, the proximal end portion 202 of the tubular body 202 occupies at least a portion of the vaginal fornix. Moreover, the proximal end portion 206 can be sized and anatomically designed to contact the walls of the vaginal canal such that the walls exert pressure onto the outer surface of the tubular body 202 to decrease the likelihood or prevent the intravaginal device 200 from slipping or otherwise substantially moving during sexual intercourse. Such a design also allows the intravaginal device 200 to maintain alignment of the second opening 212 with the external os. Generally, the tubular body 202 has an overall length of about 1.5-3.0 inches (38.1-76.2 mm). For example, embodiments of the tubular body 202 have a length of approximately 2.02.5 inches (50.8-71.1 mm).

In some embodiments, the tubular body 202 is entirely impermeable. For example, the material that forms the tubular body 202 may be impermeable to fluid penetration. As another example, the material that forms the tubular body 202 may be covered in one or more impermeable coatings (e.g., during a manufacturing process). In other embodiments, the tubular body 202 is semi-permeable. For example, the tubular body 202 may have a permeable exterior surface, while the inner surface 216 that defines the flow channel 214 is at least substantially impermeable. For example, the inner surface 216 can be coated with an impermeable coating or lined with an impermeable material. Accordingly, fluids, such as ejaculate, within the flow channel 214 may be kept separated from fluids of the intravaginal environment. As another example, the tubular body 202 may have an at least substantially impermeable exterior surface, while the inner surface 216 that defines the flow channel 214 is at least partially permeable. For instance, if the tubular body 202 is comprised of a foam material, stretching the tubular body 202 may cause the exterior surface to become a skinned surface, thereby substantially preventing fluid penetration. In some embodiments, the flow channel 214 is coated with a hydrophobic coating to allow ejaculate to flow more freely along the channel 214 and inhibit accumulation of the ejaculate within the flow channel 214. One example of a hydrophobic coating is a lubricious polytetrafluoroethylene-based (PTFE-based) hydrophobic coating.

Intravaginal devices can include various features to enhance the flow of ejaculate toward the proximal end portion and reduce the loss of ejaculate due to leakage (e.g., from the distal end portion).

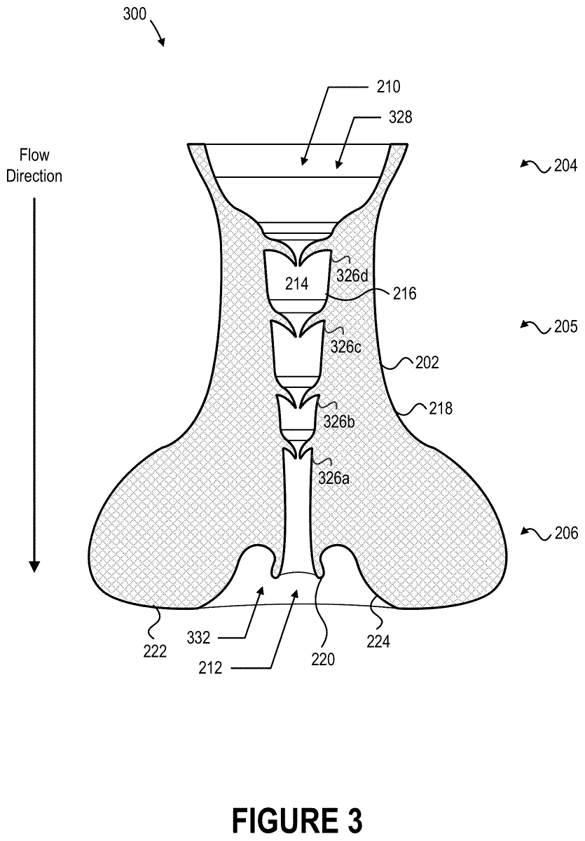

FIG. 3 is a side cross-sectional view of an intravaginal device 300 configured in accordance with various embodiments of the present technology. The intravaginal device 300 includes several features at least generally similar to the intravaginal device 200 described with reference to FIGS. 2A and 2B above. For example, the intravaginal device 300 includes the tubular body 202 having the distal end portion 204, the medial portion 205, the proximal portion 206, and the flow channel 214 extending between the first and second openings 210 and 212. In the illustrated embodiment, the intravaginal device 300 includes a depression located at either end of the tubular body 202. More specifically, the distal end portion 204 includes a first depression 328 that includes or defines the first opening 210 and is defined by a concave, tapered, or funneled surface that leads to the flow channel 214. Similar to the intravaginal device 200 of FIGS. 2A and 2B, the proximal end portion 206 includes a second depression 332 defined by a concave surface 332 and includes or defines the second opening 212.

As shown in the embodiment illustrated in FIG. 3, the surface 216 that defines the flow channel 214 includes a series of corrugations 326a-d (referred to collectively as “corrugations 326;” also referred to as “kerfs”) to inhibit ejaculate from flowing away from the cervix and leakage from the vagina. For example, the corrugations 326 are shaped in a manner to at least substantially reduce or prevent the ejaculate from flowing backward toward the distal end portion 204 away from the cervix, while allowing ejaculate flowing toward the cervix (i.e., toward the proximal end portion 206) to flow forward unimpeded. In the illustrated embodiment, the flow channel 214 includes four corrugations 326. In other embodiments, the flow channel 214 may include one corrugation, two corrugations, three corrugations, or more than four corrugations. The corrugations 326 can be spaced at evenly spaced distances along the flow channel 214 or be spaced only along selected portions of the flow channel 214. In some embodiments, the corrugations 326 can become smaller in size as they approach the proximal end portion 206 to reflect the decreasing diameter of the flow channel 214.

When the intravaginal device 300 is compressed laterally (e.g., due to a pressure applied by the vaginal walls to the outer longitudinal surface 218 of the tubular body 202), ejaculate trapped by the corrugations 326 can be forced downstream in the flow direction toward the proximal end portion 206 of the intravaginal device 300. For example, each application of pressure may cause ejaculate to travel to the next corrugation or to the depression defined by the surface 224 at the proximal end portion 206. In some embodiments, the intravaginal device 300 also includes the gasket-like structure or inner extension feature 220, which is designed to stretch into or around the external os upon an application of pressure at the distal end portion 204. For example, when pressure is applied at the distal end portion 204, the proximal end portion 206 may radially expand and the inner extension feature 220 may extend through the external os into the endocervix to more directly funnel ejaculate into the uterus. When the pressure ceases to be applied at the distal end portion 204, the extension feature 220 may return to its original shape.

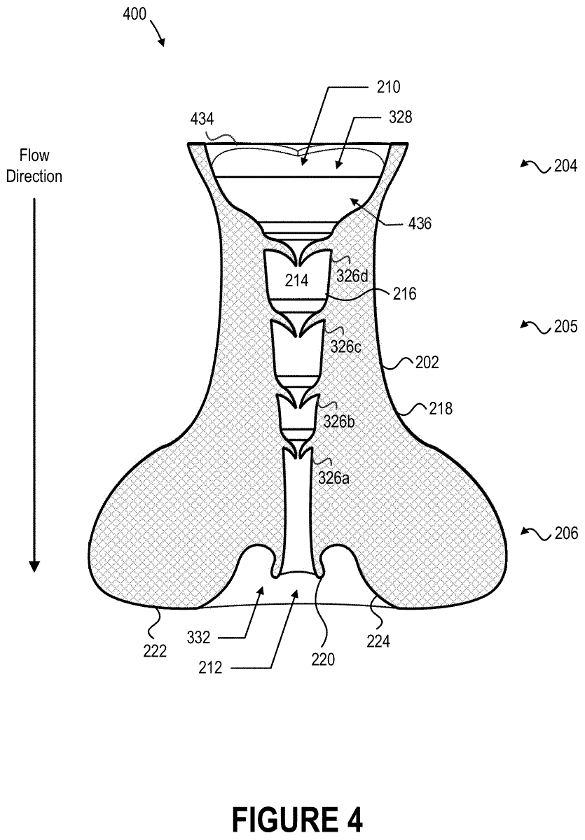

FIG. 4 is a side cross-sectional view of an intravaginal device 400 configured in accordance with embodiments of the present technology. The intravaginal device 400 includes several features at least generally similar to the intravaginal device 300 described above with reference to FIG. 3. However, the intravaginal device 400 of FIG. 4 includes a valve 434, such as a one-way valve, positioned at the distal end portion 204 of the tubular member 202 and configured to open to permit fluid (e.g., semen) to flow therethrough. For example, the valve 434 can be configured to open to permit fluid flow therethrough upon occurrence of a predetermined pressure gradient across the valve.

When pressure is applied to the distal end portion 204, the valve 434 may open to reveal the flow channel 214. Fluid (e.g., ejaculate) can then readily enter the flow channel 214 through the valve 434. The valve 434 will close to prevent backflow and/or leakage when the pressure is no longer applied. Thus, the valve 434 can ensure ejaculate that enters the flow channel 214 will only exit the intravaginal device through the second opening 212 at the proximal end portion 206. Moreover, each application of pressure may cause the ejaculate to move further down the flow channel 212 (e.g., added by the kerfs or corrugations 326).

In some embodiments, the flow channel 214 includes an expandable chamber 436 at the proximal side of the valve 434. The chamber 436 can be configured to expand to hold fluid that flows through the valve 434, and then gently return to its original shape as the fluid travels along the flow channel 214 in the flow direction.

Pressure may be applied to the distal end portion 204 and/or the sidewall 218 of the tubular body 202 by repeated applications of a physical pressure at the distal end portion 204 during intercourse or manual pulsation, vaginal contractions, or a mechanism that simulates a pumping action. For example, pressure may be applied by an external device (e.g., a blunt-end plunger or expandable balloon-type mechanism) designed to fill the chamber 436 and force fluid toward the proximal end portion 206. Thus, the intravaginal device 400 may also be used as an aid in non-natural insemination (e.g., as an alternative to other techniques such as the “turkey baster” method). To further enhance effectiveness, a lavage may be used to force any ejaculate remaining in the flow channel following sexual intercourse toward the cervix. The fluid used in the lavage may contain a protein nutrient solution (e.g., to improve/maintain health of spermatozoa in the ejaculate).

In various embodiments, the proximal end portion 206 may have an anterior side that is shorter than a posterior side to better conform to the anatomy of the vaginal cavity. For example, the anterior side may be shortened while the posterior side may be lengthened. Such a design may result in the proximal end portion 206 having a degree of offset from a longitudinal direction of a vaginal canal.

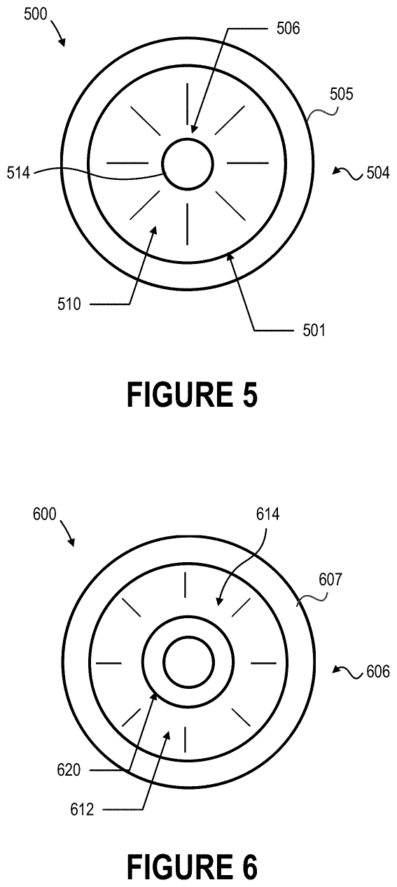

FIG. 5 is a top view of a distal end portion of an intravaginal device 500 configured in accordance with embodiments of the present technology. Similar to the intravaginal devices described above, the intravaginal device 500 includes a first opening 510 at a distal end portion 504 at which ejaculate is deposited during sexual intercourse. The ejaculate can flow through the first opening 510 into a flow channel 514, which guides the ejaculate toward the cervix. The first opening 510 may correspond to a first depression defined by a concave or other inwardly-facing surface along the distal end portion 504.

In some embodiments, the flow channel 514 includes tapered sides. Such a design allows the ejaculate to be naturally funneled toward a proximal end portion of the intravaginal device 500. In the embodiment illustrated in FIG. 5, for example, the flow channel 514 includes a distal end with a perimeter is defined by a first radius 501 and a proximal end whose perimeter is defined by a second radius 503 smaller than the first radius 501.

FIG. 6 is a bottom view of a proximal end portion 606 of an intravaginal device 600. Similar to the intravaginal devices described above, the intravaginal device 600 includes a second opening 612 at the proximal end portion 606 at which the ejaculate is presented to the external os. The second opening 612 may correspond to a second depression defined by a concave surface along the proximal end portion 606. Often, the second depression is sized to encircle the external os. Such a design can inhibit exposure of the ejaculate to the intravaginal environment, as well as inhibit migration of the ejaculate into anatomical areas other than the cervix. Some embodiments of the intravaginal device 600 include a gasket feature 620 designed to fit over or into the external os. Ejaculate exiting a flow channel 614 through the gasket feature 620 may be delivered directly to the cervix and into the uterus, or may be collected within the second depression for presentation to the external os.

As shown in FIG. 5, the distal end portion 504 can include an annular surface 505 that encircles the first depression. Similarly, as shown in FIG. 6, the proximal end portion 606 can include an annular surface 607 that encircles the second depression. In some embodiments, one or both of these annular surfaces 505, 607 are circular in shape. In other embodiments, one or both of these annular surfaces 505, 607 are elliptical in shape. For example, the front-to-back major axis of the annular surface 607 along the proximal end portion 606 may be longer than the side-to-side minor axis. In further embodiments, the annular surfaces 505, 607 can have square, rectangular, oval, irregular, and/or other suitable shapes for receiving and presenting ejaculate.

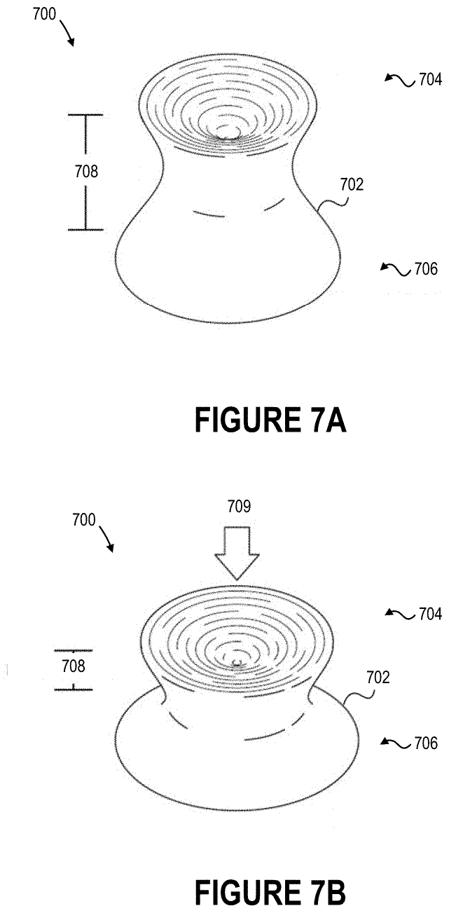

FIG. 7A is an isometric view of an intravaginal device 700 in a neutral state in accordance with embodiments of the present technology, and FIG. 7B is an isometric view of the intravaginal device 700 of FIG. 7B in a compressed state. The intravaginal device 700 can include several features generally similar to the features of the intravaginal devices described above. For example, the intravaginal device 700 can include a tubular body 702 comprised of an elastomeric or other resiliently deformable material. Thus, the tubular body 702 may include an elastic zone 708 designed to controllably deform when pressure is applied to the distal end portion 704. As shown in FIG. 7B, when pressure is applied on the distal end portion 704 in a generally proximal direction (as indicated by arrow 709), the elastic zone 708 can partially or entirely collapse downward. This downward or proximally-directed force may occur as the penis presses against the distal end portion 704. Moreover, the proximal end portion 706 may be configured to radially expand when the pressure is applied to the distal end portion 704. Upon removal of the force, the elastic zone 708 can resilient return to its original, non-collapsed state or nearly the same state as FIG. 7A.

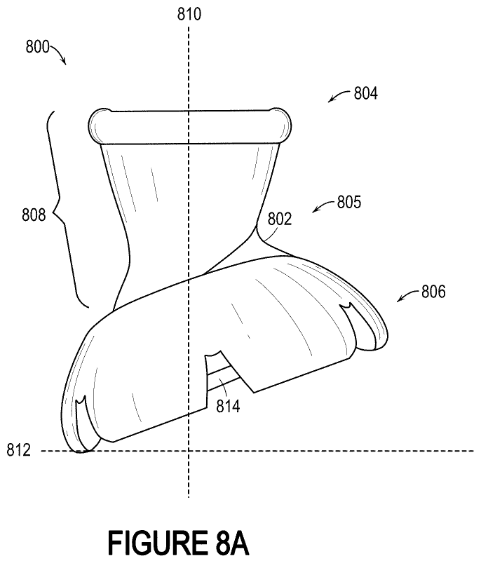

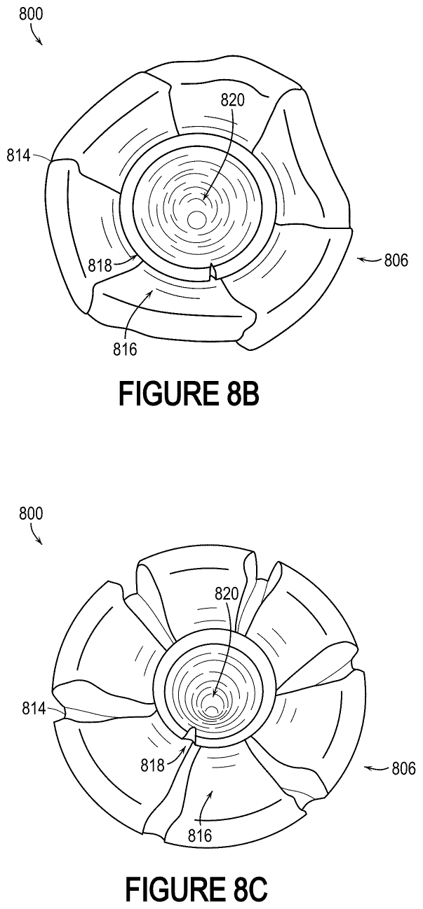

FIG. 8A is a side view of an intravaginal device 800 for enhanced natural insemination in accordance with some embodiments of the present technology. The intravaginal device 800 includes a tubular body 802 having a first or distal end portion 804, a medial portion 805, and a second or proximal end portion 806. A flow channel defined by the inner surface of the tubular body 802 can extend between the distal and proximal end portions 804 and 806. As described above, ejaculate collects at the distal end portion 804 during sexual intercourse, and then moves through the flow channel where it is presented to the external os at the proximal end portion 806.

Similar to the intravaginal device of FIG. 1, the tubular body 802 may be designed to include an elastic zone 808 (also referred to as a “crush zone”) that can be deformed under a force applied generally along a longitudinal axis 810 of the intravaginal device 800. Such force may be applied, for example, by a penis on the distal end portion 804 during sexual intercourse. Upon application of certain pressures to the distal end portion 804, the elastic zone 808 can partially collapse or otherwise deform toward the proximal end portion 806 (e.g., to accommodate a force directed downward from the distal end portion 804 toward the proximal end portion 806). In the embodiment illustrated in FIG. 8A, the proximal end portion 806 is tilted with respect to the medial portion 805 of the tubular body 802. For example, the proximal end portion 806 may be arranged at an acute angle (e.g., 15, 30, or 45 degrees) with respect to a latitudinal axis 812 that is substantially orthogonal to the longitudinal axis 810 of the intravaginal device 800.

As shown in FIG. 8A, the proximal end portion 806 of the tubular body 802 can be a fluted frustum designed to encircle the external os. The term “fluted,” as used herein, means that the corresponding structure includes structural features, such as channels, grooves, or depressions, that promote collapse upon experiencing pressure. Here, for example, the proximal end portion 806 includes six structural features spaced around its perimeter.

Such a design allows the proximal end portion 806 of the intravaginal device 800 to be in a compressed state when inserted into the vaginal cavity, as shown in FIG. 8B. After the intravaginal device 800 has been inserted into the vaginal cavity, the proximal end portion 806 may revert into its natural, expanded state. FIG. 8C is a bottom view of the proximal end portion 806 of the intravaginal device 800 of FIG. 8A in a natural state. When a force is applied generally along the longitudinal axis 810 of the intravaginal device 800, the proximal end portion 806 may widen into an expanded state. In the expanded state, segments of the proximal end portion 806 defined by the structural features may flare out to widen the opening of the proximal end portion 806. Accordingly, such a design may allow the proximal end portion 806 of the tubular body 802 to collapse for easier installation, as well as better accommodate a larger variety of anatomical differences.

Similar to the intravaginal devices described above, the intravaginal device 800 includes an opening 816 at the proximal end portion 806 at which ejaculate is presented to the external os. The opening 816 may correspond to a depression defined by a concave surface along the proximal end portion 806. As noted above, the concave surface may include structural features 814 that enable the depression to readily accommodate different anatomies. Some embodiments of the intravaginal device 800 include a gasket feature 818 designed to fit over or into the external os. Ejaculate exiting a flow channel 820 through the gasket feature 818 may be delivered directly to the cervix and into the uterus, or may be collected within the depression for presentation to the external os.

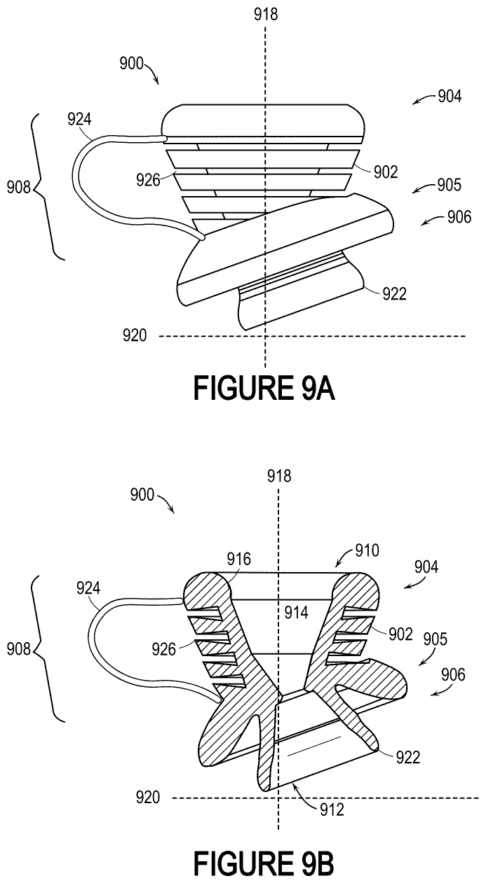

FIG. 9A is a side view of an intravaginal device 900 for enhanced insemination configured in accordance with embodiments of the present technology, and FIG. 9B is a side cross-sectional view of the intravaginal device 900 of FIG. 9A. The intravaginal device 900 includes several features at least generally similar to the features of the intravaginal devices 100 and 800 described with reference to FIGS. 1 and 8A-8C above. For example, the intravaginal device 900 includes a tubular body 902 having a distal end portion 904 configured to be positioned proximate to the vaginal opening, a proximal end portion 906 opposite the distal end portion 904 and configured to be positioned proximate to the cervix, a medial portion 905 spacing the distal and proximal portions 904 and 906 apart from each other, and a flow channel 914 defined by an inner surface 916 of the tubular body 902 and configured to transport ejaculate from the distal end portion 904 to the proximal end portion 906 where it can be presented to the external os of the cervix. The tubular body 902 can also include an elastic zone or region 908 that can at least partially collapse under pressure applied to the distal end portion 904 during intercourse. Much like the intravaginal device 800 described with reference to FIGS. 8A-8C, the proximal end portion 906 can be tilted with respect to the medial portion 905 of the tubular body 902. For example, the proximal end portion 906 may be arranged at an acute angle (e.g., an angle of 15, 30, or 45 degrees) with respect to a latitudinal axis 920 that is substantially orthogonal to the longitudinal axis 918 of the intravaginal device 900.

As shown in FIG. 9B, the intravaginal device 900 can further include a first opening 910 at the distal end portion 904 at which ejaculate can be naturally deposited during sexual intercourse or artificially deposited (e.g., via a syringe) and a second opening 912 at the proximal end portion 906 at which the ejaculate can be presented to the external os. When ejaculate is deposited at the first opening 910, it can travel to the second opening 912 through the flow channel 914. The first opening 910 and/or the second opening 912 can be defined by or extend from depressions at the distal end portion 904 and/or the proximal end portion 906, respectively. In the embodiment illustrated in FIG. 9B, for example, the proximal end portion 906 includes a depression defined by an enlarged gasket feature 922, and the second opening 912 is within or defined by the depression. As shown in FIG. 9B, the proximal end portion 906 can include a shortened frustum (also referred to as an “anchor cone”) designed such that its proximal end surface may not contact the ectocervix and/or the vaginal wall around the ectocervix.

As noted above, the tubular body 902 can include an enlarged gasket structure 922 at the proximal end portion 906 that defines the second opening 912. The enlarged gasket feature can be sized and shaped to fit over or around the external os. In some embodiments, the enlarged gasket structure 922 has an inner diameter of approximately 1.0-1.25 inches (25.4-31.75 mm). When pressure is applied to the intravaginal device 900 at the distal end portion 904, the enlarged gasket structure 922 may become partially compressed, thereby forcing ejaculate near or into the external os of the cervix.

In some embodiments, the intravaginal device 900 includes a removal cord 924. Here, the ends of the removal cord 924 are secured at the distal end portion 904 and the proximal end portion 906. However, in other embodiments, both ends of the removal cord 924 are secured at the distal end portion 904, medial portion 905, or proximal end portion 906. The removal cord 924 enables the intravaginal device 900 to be easily removed from the vaginal cavity following intercourse. The removal cord 924 may be coated in an impermeable coating. For example, the removal cord 924 may be coated with a hydrophobic coating to inhibit absorption of fluids present in the vaginal cavity.

As shown in FIGS. 9A and 9B, the tubular body 902 can include axial corrugations 926 arranged along the distal end portion 904 and/or the medial portion 905. The axial corrugations 926 are evenly spaced along the length of the distal end portion 904 and/or the medial portion 905 in some embodiments. In other embodiments, the density of the axial corrugations 926 varies along the length of the distal end portion 904 and/or the medial portion 905. For example, the density of the axial corrugations 926 may increase as the distance from the distal end portion 904 increase. The axial corrugations 926 can be tapered to improve circumferential strength while continuing to allow longitudinal collapse under pressure applied along the longitudinal axis 918.

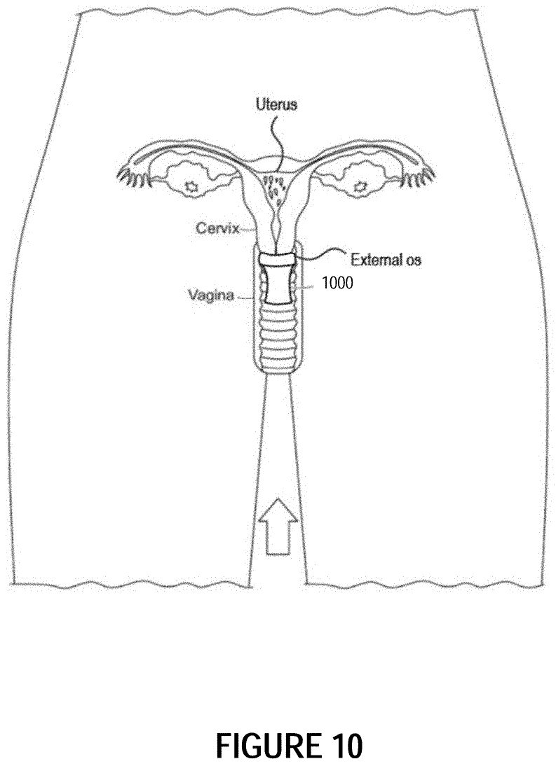

FIG. 10 is an illustration of an intravaginal device 1000 deployed within the anatomy in accordance with embodiments of the present technology. The intravaginal device 1000 can include several features at least generally similar to the features of the intravaginal devices described above with reference to FIGS. 1-9B. Initially, the intravaginal device 1000 can be partially compressed and placed within the vaginal cavity before sexual intercourse begins. In some embodiments, the intravaginal device 1000 is sterilized (e.g., during a manufacturing process) prior to deployment. As noted above, the intravaginal device 1000 can be designed to fit snugly within the vaginal cavity during sexual intercourse. For example, the intravaginal device 1000 may be sized to have a larger diameter or cross-section area than the vaginal canal such that, when positioned in the desired location, the intravaginal device expands to contact and press against the walls of the vaginal canal when deployed. In some embodiments, the intravaginal device 1000 is columnar in form with outwardly tapered sides such that a proximal end portion nearest the external os and cervix occupies at least part of the fornix.

During sexual intercourse, ejaculate is collected by the intravaginal device 1000 at the distal end portion, guided through the intravaginal device 1000 along the flow channel, and then presented to the external os at the proximal end portion. The intravaginal device 1000 may remain in the vaginal cavity for a prescribed duration of time (e.g., several minutes to several hours) following sexual intercourse. After the prescribed amount of time has elapsed, the intravaginal device 1000 can be removed by grasping the distal end portion and pulling until the proximal end portion clears the vaginal opening.

In some embodiments, the intravaginal device 1000 serves as a passive spermatozoa concentration mechanism. The intravaginal device may be “passive” in the sense that ejaculate can be guided through the flow channel without the use of any moving parts. Instead, ejaculate can naturally flow through the flow channel due to gravity or another natural means (e.g., pressure or swimming). In other embodiments, the intravaginal device 1000 serves as a semi-passive spermatozoa concentration mechanism. The intravaginal device may be “semi-passive” in the sense that it may include features (e.g., corrugations, valves, collection chambers) that facilitate the movement of ejaculate responsive to external applications of pressure to the distal end portion and/or the sidewalls (e.g., occurring naturally during intercourse, manual, and/or mechanical).

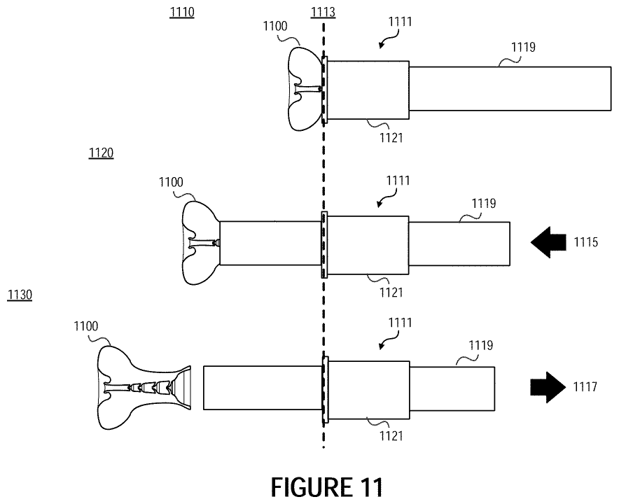

Often, a user will insert an intravaginal device into the vaginal cavity using her hand. However, in some instances, the user may find it useful to insert the intravaginal device into the vaginal cavity using an installation mechanism or delivery device. FIG. 11 is a series of side views illustrating a procedure for inserting an intravaginal device 1100 into the vaginal canal using an installation mechanism or delivery device 1111 to which the intravaginal device 1100 is detachably connected. The top view 1110 depicts a first or initial position of the intravaginal device 1100 upon insertion into the vaginal cavity, where the broken line 1113 indicates the exterior surface of the vaginal opening. In the center image 1120, the delivery device 1111 is inserted a desired depth into the vaginal opening and moves to a second position in which the delivery device 1111 is actuated (e.g., manually by the user) in a proximal direction toward the external os (as indicated by arrow 1115). Upon actuation, a shaft 1119 of the delivery device 1111 may be longitudinally actuated to extend through and out of an exterior shell 1121. As shown in the bottom image 1130, the shaft 1119 can be further actuated until the proximal end portion of the intravaginal device 1100 contacts the vaginal walls near the external os. Once at the desired position within the vagina, the delivery device 1111 can release the intravaginal device 1100, and the delivery device 1111 can be removed from the body (as indicated by arrow 1117).

The same delivery device 1111 or a similar mechanism may be used to repeatedly apply pressure at the distal end portion of the intravaginal device 1100 to simulate a pumping action. For example, pressure may be applied to the depression at the distal end portion to force fluid along the flow channel toward the proximal end portion. Examples of such devices include blunt-end plungers, expandable balloon-type mechanisms, and similar devices that can exert force on the distal end portion of the intravaginal device 1100.

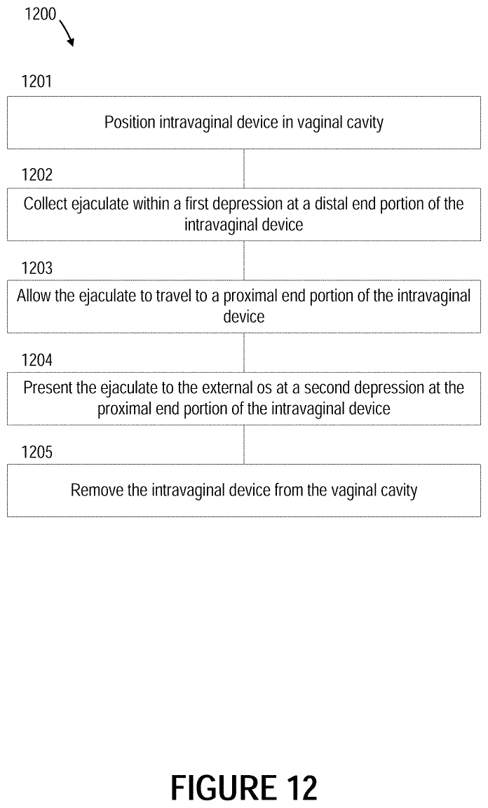

FIG. 12 is a block diagram illustrating a method 1200 for using an intravaginal device (e.g., the intravaginal devices described above) to enhance the likelihood of a pregnancy through natural insemination in accordance with embodiments of the present technology. Initially, a user can position the intravaginal device in the vaginal cavity (step 1201). In some embodiments the intravaginal device is inserted using a delivery device (e.g., delivery device 1102 of FIG. 11), while in other embodiments the intravaginal device is inserted using a hand.

During sexual intercourse, ejaculate can be collected within a first depression defined by a concave surface along a distal end portion of the intravaginal device (step 1202). Thereafter, the intravaginal device can allow the ejaculate to travel from the distal end portion to a proximal end portion of the intravaginal device (step 1203). In some embodiments, the ejaculate naturally flows from the distal end portion to the proximal end portion along a flow channel having tapered sides. Additionally or alternatively, the ejaculate may be forced along the flow channel due to an application of pressure along the outer surface of the intravaginal device. For example, an application of pressure at the first depression along the distal end portion may cause the ejaculate to travel toward the proximal end portion.

The ejaculate can then be presented to the external os within a second depression defined by a concave surface along the proximal end portion of the intravaginal device (step 1204). The intravaginal device may remain in the vaginal cavity until substantially all ejaculate has been guided into the cervix or until a prescribed amount of time (e.g., several minutes to several hours) has elapsed. Following the conclusion of either of these events, the intravaginal device can be removed from the vaginal cavity (step 1205). For example, the user may remove the intravaginal device by grasping the distal end portion and pulling until the proximal end portion clears the vaginal opening.

Unless contrary to physical possibility, it is envisioned that the steps described above may be performed in various sequences and combinations. For example, the intravaginal device may simultaneously collect ejaculate at the distal end portion, funnel ejaculate toward the proximal end portion along the flow channel, and present ejaculate at the proximal end portion.

Other steps may also be included in some embodiments. For example, some embodiments of the intravaginal device are configured to be wetted with a lubricant for easier deployment. Thus, a user may apply a lubricant to the exterior surface of the intravaginal device prior to insertion into the vaginal cavity. In some embodiments, the intravaginal device is pre-lubricated. Thus, the intravaginal device may include at least some lubrication along the exterior surface while still in the packaging.

The present technology may also be used in conjunction with other fertility therapies and/or devices. For example, after ejaculate has been guided into/near the cervix using an intravaginal device, a user may remove the intravaginal device and insert a cap or a plug designed to prevent ejaculate from leaking from the cervix into the vaginal cavity.

### EXAMPLES

Several aspects of the present technology are set forth in the following examples.

1. An intravaginal device comprising:
a tubular body comprising—
   a first portion having a first concave surface that defines a first depression at which to collect ejaculate during intercourse,
   a second portion having a second concave surface that defines a second depression at which to present the ejaculate to an external orifice of a cervix, and
   a flow channel defined by an inner surface of the tubular body extending between the first and second depressions, wherein the flow channel is configured to allow the ejaculate to travel from the first depression to the second depression for presentation to the external os of the cervix.
      2. The intravaginal device of example 1 wherein the second depression is sized to encircle the external orifice of the cervix.
      3. The intravaginal device of example 1 wherein the tubular body further comprises an outer surface, and wherein the outer surface is configured to enclose the first and second depressions and the flow channel to inhibit exposure of the ejaculate to an intravaginal environment.
      4. The intravaginal device of example 1, further comprising:
a corrugation defined along the inner surface that defines the flow channel,
   wherein the corrugation prevents backflow of the ejaculate toward the first depression.
      5. The intravaginal device of example 4 wherein:
the corrugation is one of multiple corrugations defined along the inner surface, and
the multiple corrugations enable the ejaculate to travel along the flow channel in increments each time a pressure is applied near the first depression of the tubular body.
   6. The intravaginal device of example 1 wherein the tubular body further comprises an outer surface, and wherein the outer surface is configured to be wetted with a lubricant for easier deployment.
   7. The intravaginal device of example 1 wherein the inner surface comprises a hydrophobic coating to inhibit accumulation of the ejaculate.
   8. The intravaginal device of example 1, further comprising:
a valve disposed proximate to the first depression,
wherein the valve is configured to open to permit fluid flow therethrough upon occurrence of a predetermined pressure gradient across the valve.
   9. The intravaginal device of example 1 wherein the tubular body further comprises an outer surface, and wherein the outer surface defines a distal end portion in the form of a concave cylinder and a proximal end portion in the form of a hemisphere.
   10. The intravaginal device of example 1 wherein the tubular body further comprises an outer surface, and wherein the outer surface defines a distal end portion in the form of a tapered cylinder and a proximal end portion in the form of a hemisphere.
   11. The intravaginal device of example 1 wherein the flow channel is a tapered channel designed to guide the ejaculate from the first depression to the second depression along the flow channel.
   12. The intravaginal device of example 1 wherein the second depression includes a gasket that is sized to enclose the external orifice.
   13. The intravaginal device of example 1 wherein the second portion of the tubular body includes multiple channels that define multiple segments, and wherein the multiple segments are configured to flare outward when a pressure is applied near the first depression of the tubular body.
   14. The intravaginal device of example 1 wherein the tubular body further comprises an elastic zone configured to deform when a pressure is applied near the first depression of the tubular body along a longitudinal axis of the intravaginal device, and wherein the second portion is tilted with respect to the elastic zone at an angle of at least 15 degrees with respect to a latitudinal axis that is substantially orthogonal to the longitudinal axis.
   15. The intravaginal device of example 1, further comprising:
a string having opposing ends attached to the first and second portions of the tubular body.
   16. The intravaginal device of example 1, further comprising:
a corrugation defined along an outer surface of the tubular body,
   wherein the corrugation encourages partial collapse of the tubular body when a pressure is applied near the first depression of the tubular body.
      17. The intravaginal device of example 16 wherein the corrugation is one of multiple corrugations defined along the outer surface of the tubular body.
      18. An intravaginal device comprising:
a tubular body having—
   a distal end portion at which to collect ejaculate during intercourse,
   a proximal end portion at which to present the ejaculate to an external orifice of a cervix, and
   a flow channel through which the ejaculate travels from the distal end portion to the proximal end portion; and
a valve disposed at the distal end portion of the tubular body,
   wherein the valve is configured to open to permit fluid flow therethrough upon occurrence of a predetermined pressure gradient across the valve.
      19. The intravaginal device of example 18 wherein the flow channel includes an expandable chamber disposed on a proximal side of the valve.
      20. The intravaginal device of example 18 wherein the tubular body is comprised of an elastomeric material capable of being deformed under pressure during sexual intercourse.
      21. The intravaginal device of example 20 wherein, upon placement of a predetermined pressure at the distal end portion of the tubular body, a portion of proximal end portion of the tubular body is configured to stretch past the external orifice of the cervix.
      22. The intravaginal device of example 18 wherein the tubular body is at least 1.5 inches in length and no more than 3 inches in length.
      23. The intravaginal device of example 18 wherein an outer surface of the tubular body is sized to contact the walls of a vaginal canal when deployed.
      24. The intravaginal device of example 18 wherein the tubular body is comprised of a semi-permeable sponge material or a semi-permeable foam material.
      25. The intravaginal device of example 24 wherein the flow channel is defined by an inner surface of the tubular body that is impermeable to fluid.
      26. The intravaginal device of example 18 wherein:
the proximal end portion of the tubular body includes a concave depression sized to encircle the external orifice of the cervix, and
the concave depression has an anterior side that is shorter than a posterior side, thereby resulting in the proximal end portion of the tubular body having a degree of offset from a longitudinal direction of a vaginal canal when deployed.
   27. A method for delivering ejaculate produced during sexual intercourse to an external orifice of a cervix while inhibiting exposure to an intravaginal environment, the method comprising:
collecting the ejaculate within a first depression at a distal end portion of an intravaginal device;
guiding the ejaculate from the first depression to a second depression at a proximal end portion of the intravaginal device along a flow channel; and
presenting the ejaculate to the external orifice at the second depression,
   wherein a perimeter of the second depression is defined by a surface of the intravaginal device that is configured to maintain contact with a vaginal wall to inhibit leakage of the ejaculate into the intravaginal environment, and
   wherein the perimeter of the second depression is sized to encircle the external orifice of the cervix.
      28. The method of example 27, further comprising:
allowing the intravaginal device to be positioned within the intravaginal environment; and
following an elapse of a certain amount of time, allowing the intravaginal device to be removed from the intravaginal environment.
   29. The method of example 28 wherein the intravaginal device is positioned within the intravaginal environment using a delivery device to which the intravaginal device is detachably connected.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An intravaginal device comprising:
a tubular body comprising—
a first portion having a first concave surface that defines a first depression at which to collect ejaculate during intercourse,
a second portion having a second concave surface that defines a second depression at which to present the ejaculate to an external orifice of a cervix, and
a flow channel defined by an inner surface of the tubular body extending between the first and second depressions, wherein the flow channel is configured to allow the ejaculate to travel from the first depression to the second depression for presentation to the external os of the cervix.

2. The intravaginal device of claim 1 wherein the second depression is sized to encircle the external orifice of the cervix.

3. The intravaginal device of claim 1 wherein the tubular body further comprises an outer surface, and wherein the outer surface is configured to enclose the first and second depressions and the flow channel to inhibit exposure of the ejaculate to an intravaginal environment.

4. The intravaginal device of claim 1, further comprising:
a corrugation defined along the inner surface that defines the flow channel,
wherein the corrugation prevents backflow of the ejaculate toward the first depression.

5. The intravaginal device of claim 4 wherein:
the corrugation is one of multiple corrugations defined along the inner surface, and
the multiple corrugations enable the ejaculate to travel along the flow channel in increments each time a pressure is applied near the first depression of the tubular body.

6. The intravaginal device of claim 1 wherein the tubular body further comprises an outer surface, and wherein the outer surface is configured to be wetted with a lubricant for easier deployment.

7. The intravaginal device of claim 1 wherein the inner surface comprises a hydrophobic coating to inhibit accumulation of the ejaculate.

8. The intravaginal device of claim 1, further comprising:
a valve disposed proximate to the first depression,
wherein the valve is configured to open to permit fluid flow therethrough upon occurrence of a predetermined pressure gradient across the valve.

9. The intravaginal device of claim 1 wherein the tubular body further comprises an outer surface, and wherein the outer surface defines a distal end portion in the form of a concave cylinder and a proximal end portion in the form of a hemisphere.

10. The intravaginal device of claim 1 wherein the tubular body further comprises an outer surface, and wherein the outer surface defines a distal end portion in the form of a tapered cylinder and a proximal end portion in the form of a hemisphere.

11. The intravaginal device of claim 1 wherein the flow channel is a tapered channel designed to guide the ejaculate from the first depression to the second depression along the flow channel.

12. The intravaginal device of claim 1 wherein the second depression includes a gasket that is sized to enclose the external orifice.

13. The intravaginal device of claim 1 wherein the second portion of the tubular body includes multiple channels that define multiple segments, and wherein the multiple segments are configured to flare outward when a pressure is applied near the first depression of the tubular body.

14. The intravaginal device of claim 1 wherein the tubular body further comprises an elastic zone configured to deform when a pressure is applied near the first depression of the tubular body along a longitudinal axis of the intravaginal device, and wherein the second portion is tilted with respect to the elastic zone at an angle of at least 15 degrees with respect to a latitudinal axis that is substantially orthogonal to the longitudinal axis.

15. The intravaginal device of claim 1, further comprising:
a string having opposing ends attached to the first and second portions of the tubular body.

16. The intravaginal device of claim 1, further comprising:
a corrugation defined along an outer surface of the tubular body,
wherein the corrugation encourages partial collapse of the tubular body when a pressure is applied near the first depression of the tubular body.

17. The intravaginal device of claim 16 wherein the corrugation is one of multiple corrugations defined along the outer surface of the tubular body.

18. An intravaginal device comprising:
a tubular body having—
a distal end portion at which to collect ejaculate during intercourse,
a proximal end portion at which to present the ejaculate to an external orifice of a cervix, and
a flow channel through which the ejaculate travels from the distal end portion to the proximal end portion; and
a valve disposed at the distal end portion of the tubular body,
wherein the valve is configured to open to permit fluid flow therethrough upon occurrence of a predetermined pressure gradient across the valve.

19. The intravaginal device of claim 18 wherein the flow channel includes an expandable chamber disposed on a proximal side of the valve.

20. The intravaginal device of claim 18 wherein the tubular body is comprised of an elastomeric material capable of being deformed under pressure during sexual intercourse.

21. The intravaginal device of claim 20 wherein, upon placement of a predetermined pressure at the distal end portion of the tubular body, a portion of proximal end portion of the tubular body is configured to stretch past the external orifice of the cervix.

22. The intravaginal device of claim 18 wherein the tubular body is at least 1.5 inches in length and no more than 3 inches in length.

23. The intravaginal device of claim 18 wherein an outer surface of the tubular body is sized to contact the walls of a vaginal canal when deployed.

24. The intravaginal device of claim 18 wherein the tubular body is comprised of a semi-permeable sponge material or a semi-permeable foam material.

25. The intravaginal device of claim 24 wherein the flow channel is defined by an inner surface of the tubular body that is impermeable to fluid.

26. The intravaginal device of claim 18 wherein:
the proximal end portion of the tubular body includes a concave depression sized to encircle the external orifice of the cervix, and
the concave depression has an anterior side that is shorter than a posterior side, thereby resulting in the proximal end portion of the tubular body having a degree of offset from a longitudinal direction of a vaginal canal when deployed.

27. A method for delivering ejaculate produced during sexual intercourse to an external orifice of a cervix while inhibiting exposure to an intravaginal environment, the method comprising:
collecting the ejaculate within a first depression at a distal end portion of an intravaginal device;
guiding the ejaculate from the first depression to a second depression at a proximal end portion of the intravaginal device along a flow channel; and
presenting the ejaculate to the external orifice at the second depression,
wherein a perimeter of the second depression is defined by a surface of the intravaginal device that is configured to maintain contact with a vaginal wall to inhibit leakage of the ejaculate into the intravaginal environment, and
wherein the perimeter of the second depression is sized to encircle the external orifice of the cervix.

28. The method of claim 27, further comprising:
allowing the intravaginal device to be positioned within the intravaginal environment; and
following an elapse of a certain amount of time, allowing the intravaginal device to be removed from the intravaginal environment.

29. The method of claim 28 wherein the intravaginal device is positioned within the intravaginal environment using a delivery device to which the intravaginal device is detachably connected.
